(19) United States
(12) Patent Application Publication
(10) Pub. No.: US 2021/0151145 A1
(43) Pub. Date: May 20, 2021

(54) SYSTEMS AND METHODS FOR EMERGENCY MEDICAL MONITORING

(71) Applicant: Poudre Valley Health Care, Inc., Loveland, CO (US)

(72) Inventors: Julie Dunn, Fort Collins, CO (US); Ben Walker, Loveland, CO (US); Katherine Corey, Loveland, CO (US)

(73) Assignee: Poudre Valley Health Care, Inc., Loveland, CO (US)

(21) Appl. No.: 17/145,111

(22) Filed: Jan. 8, 2021

Related U.S. Application Data
(63) Continuation of application No. 17/105,754, filed on Nov. 27, 2020.
Application No. 17/105,754 is a continuation of application No. 15/524,007, filed on May 3, 2017, now abandoned.
(60) Provisional application No. 62/076,248, filed on Nov. 6, 2014.

(51) Int. Cl. G16H 10/60; G16H 40/67; G16H 40/20; G16H 15/00

(52) U.S. Cl. CPC G16H 10/60; G16H 40/67; G16H 10/20; G16H 15/00; G16H 40/20

(57) ABSTRACT

Implementations described and claimed herein provide systems and methods for emergency medical monitoring. In one implementation, one or more workflows are generated using a patient monitor executed by at least one server. The one or more workflows are configured for monitoring the patient during an emergency medical procedure. Patient medical information corresponding to an emergency medical event is captured using the one or more workflows. The patient medical information is captured using at least one user device in communication with the at least one server over a network. A patient medical summary is generated by extracting and collating the patient medical information, and the patient medical summary is output for presentation on at least one display in communication with the at least one server over the network.

### CROSS-REFERENCE TO RELATED APPLICATION

The present application is a continuation of U.S. application Ser. No. 17/105,754 filed Nov. 27, 2020, which application is a continuation of U.S. application Ser. No. 15/524,007 filed May 3, 2017, which application is a national phase of PCT Application No. PCT/US2015/059581 filed Nov. 6, 2015, which application claims priority to U.S. Provisional Application No. 62/076,248, entitled “Systems and Methods for Recording and Displaying Patient Information” and filed on Nov. 6, 2014. All of the above-referenced applications are incorporated by reference herein in their entirety.

### TECHNICAL FIELD

Aspects of the present disclosure relate to systems and methods for monitoring emergency medical events and more particularly to real-time communication and monitoring during acute trauma, cardiac resuscitation, and/or other emergency medical events.

### BACKGROUND

Response to an emergency medical event, such as acute trauma, cardiac resuscitation, and/or other serious injuries generally involves many swift actions taken by separate yet interdependent emergency medical teams. For example, an emergency medical technician (EMT) arrives on scene of the emergency medical event and takes any actions necessary to treat the patient prior to arrival at an emergency medical location, such as a hospital. Facilitating communication between these teams to ensure all actions taken by the different teams are documented and understood to continue or initiate an emergency medical procedure to address and treat the results of the emergency medical event is often challenging. For example, there are generally thousands of data points corresponding to a trauma event that lasts approximately 30-45 minutes. To provide the best care possible and avoid preventable deaths, these data points need to be captured and communicated to the emergency medical team members. Conventionally, there is a verbal handoff between the EMT team and the medical personnel at the emergency medical location, which often includes approximately 15 people. Each of these team members may be assigned a task that is directly or indirectly dependent on a task another team member is responsible for. Accordingly, accurate capture of information during the handoff and the emergency medical procedure, as well as communication regarding a status of various tasks, is difficult given the chaos often involved in emergency medical procedures.

It is with these observations in mind, among others, that various aspects of the present disclosure were conceived and developed.

### SUMMARY

Implementations described and claimed herein address the foregoing problems, among others, by providing systems and methods for emergency medical monitoring. Other implementations are also described and recited herein. Further, while multiple implementations are disclosed, still other implementations of the presently disclosed technology will become apparent to those skilled in the art from the following detailed description, which shows and describes illustrative implementations of the presently disclosed technology. As will be realized, the presently disclosed technology is capable of modifications in various aspects, all without departing from the spirit and scope of the presently disclosed technology. Accordingly, the drawings and detailed description are to be regarded as illustrative in nature and not limiting.

### BRIEF DESCRIPTION OF THE DRAWINGS

FIG. 1 shows an example patient monitoring system, including a patient monitor running on a computer server, computing device, or other device coupled with a network, for real-time communication and monitoring of a patient during an emergency medical event.

FIG. 2 illustrates an example patient medical information user interface executed by the patient monitor and running on a scribe device for recording patient information during an emergency medical event.

FIG. 3 shows an example pre-patient arrival user interface for capturing data pertaining to a time prior to arrival of the patient at an emergency medical location, such as a hospital.

FIG. 4 is an example patient assessment user interface for capturing data providing an assessment of the patient after arrival at the emergency medical location.

FIG. 5 illustrates an example interventions user interface for recording and monitoring any medical interventions or other inputs to the patient during an emergency medical procedure at the emergency medical location.

FIG. 6 shows an example patient products user interface for recording and monitoring any orders, fluids, blood products, medications, or other products provided to the patient during the emergency medical procedure.

FIG. 7 illustrates an example narrative user interface for capturing and monitoring vital signs for the patient and recording associated narratives during the emergency medical procedure.

FIG. 8 is an example disposition user interface for recording dispositions of various aspects of the emergency medical procedure and/or event.

FIG. 9 shows an example military trauma user interface for recording and monitoring an emergency medical event in a military environment.

FIG. 10 illustrates an example patient medical information summary presented on a display during an emergency medical procedure.

FIG. 11 shows an example patient medical information user interface executed by the patient monitor and running on a physician device for recording patient information and accessing reference information during an emergency medical event.

FIG. 12 is an example computing system that may be specifically configured to implement the various systems and methods discussed herein.

### DETAILED DESCRIPTION

Aspects of the present disclosure generally involve systems and methods for emergency medical monitoring. In one particular aspect, patient information corresponding to an emergency medical procedure is captured, manually and/or automatically, and extracted, collated, and output for selective presentation on a display. A patient monitor compiles and simplifies a complex set of workflows that determine the critical elements essential for display to improve trauma awareness, resulting in more timely interventions during fast paced critical situations that will prevent harm and save lives. Further, the patient monitor provides an electronic process for capture of the patient information, reducing time expended documenting an emergency medical procedure and reducing transcription errors by pre-populating forms with the captured data, as well as pre-populating downstream forms for review and approval. Additionally, the patient monitor parses and stores the patient information in a database or storage cluster such that it may be leveraged for medical intelligence, through a pre-population of downstream reports, including billing, registries, internal reviews of trauma scenarios, statistics, and/or the like.

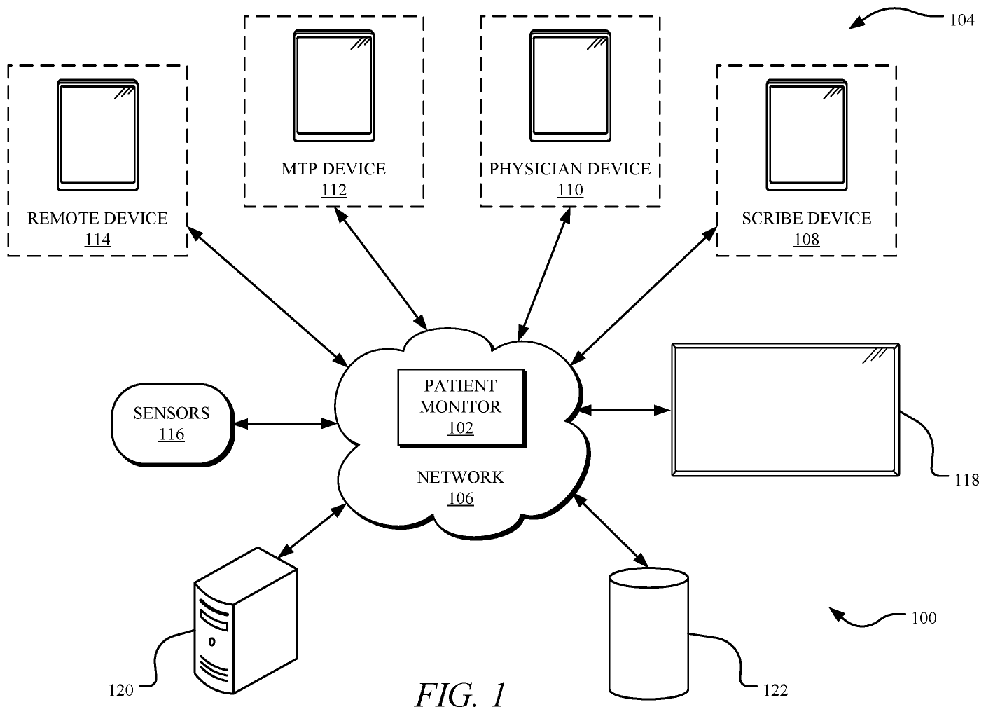

To begin a detailed description of an example patient monitoring system 100 for real-time communication and monitoring of a patient during an emergency medical event, reference is made to FIG. 1. In one implementation, a user, such as a member of an emergency medical procedure team, accesses and interacts with a patient monitor 102 using a user device 104 via a network 106.

The user device 104 is generally any form of computing device capable of interacting with the network 106, such as a personal computer, terminal, workstation, portable computer, mobile device, smartphone, tablet, multimedia console, and the like. In one implementation, the user devices 104 include a scribe device 108, a physician device 110, an massive transfusion protocol (MTP) device 112, and/or a remote device 114. The devices 108-114 may be, for example, a tablet or other mobile device for capturing and inputting data pertaining to an emergency medical event in various environments. The network 106 and/or the user devices 104 further communicate with one or more sensors 116, a display 118, at least one server 120, one or more databases 122, and/or other computing or data storage devices or computing units described herein for implementing the patient monitor 102 and other services, applications, or modules in the patient monitoring system 100.

In one implementation, the patient monitoring system 100 includes the server 120 hosting a website or an application that the user may visit to access the patient monitor 102 and/or other network components. The server 120 may be a single server, a plurality of servers with each such server being a physical server or a virtual machine, or a collection of both physical servers and virtual machines. In another implementation, a cloud hosts one or more components of the patient monitoring system 100. The user devices 104, the server 120, and other resources connected to the network 106 may access one or more other servers to access to one or more websites, applications, web services interfaces, storage devices, computing devices, or the like that are used for patient monitoring and/or medical intelligence generation. The server 120 may also host a search engine that the patient monitor 102 uses for accessing, searching for, and modifying patient data, medical intelligence, and other data.

In one implementation, patient information pertaining to an emergency medical event is captured using one or more of the user devices 104. For example, the remote device 114 may be used to capture patient information pertaining to the emergency medical event prior to the patient's arrival at an emergency medical location. The remote device 114 may automatically capture such pre-patient arrival data using the one or more sensors 116 (e.g., ECG, heart rate, blood pressure, oxygen, carbon dioxide, or other sensors) or manually capture the data through input by an emergency medical professional, such as an EMT. The pre-patient arrival data may be used to pre-populate various workflows presented on the scribe device 108, as detailed herein. Once the patient arrives at the emergency medical location, the scribe device 108 is used to capture patient data pertaining to the emergency medical procedure, which may then be used to pre-populate forms, such as a Physician History and Physical Examination form, for review and approval by the physical, for example, using the physical device 110. Where an MTP is performed during the emergency medical procedure, patient data pertaining to the MTP may be captured using the MTP device 112. In addition to automatic capture, the patient data may be captured using various manual inputs detailed herein, including, without limitation, tactile, audio, and/or using a visual display. For example, the patient data may be input using a touchscreen on one of the user devices 104 and/or using voice input.

The captured patient data is extracted, collated, and output for selective presentation on the display 118. In one implementation, patient data critical to providing optimal care for the patient or otherwise to enhance situational awareness of and communication among the medical team during the emergency medical procedure is presented with the display 118. For example, fluids, drugs, and interventions administered to the patient and vital signs may be presented with the display 118 for quick reference. In one particular implementation, the display 118 generates graphical user interfaces using jPanels, jFrames, and jTables with a design tree structure. The patient monitor 102 may generate alerts indicating a critical situation for output via the user devices 104, the display 118, and/or other computing devices.

In one implementation, automated inputs and outputs and non-automated inputs may be provided to the scribe device 108 and/or other user devices 104 and to the display 118. In one example, automated inputs may include vital signs (e.g., heart rate, blood pressure, and oxygen saturations), medications, fluids, and blood. Automatic outputs may include urine output and chest tube output. In one example, non-automated inputs may include event history, pre-hospital interventions, hospital interventions (e.g., endotracheal tube, intravenous lines, chest tubes, foley, DPL, FAST, etc.), and physical exam findings. In one implementation, the displays 118 may located in multiple key areas, such as CT scan, operating room, intensive care unit. Thus, the patient monitoring system 100 may be configured to enable acquisition and display of patient data from a variety of inputs, provide alerts based upon physiologic status of patient, provide results of pertinent lab data, enable modifiable trauma nursing flow sheet output, and provide automatic download to electronic health records. In this manner, the patient monitoring system 100 may improve patient outcomes by improving situational awareness, strengthening communication, and the like.

The patient monitor 102 parses, tags, and stores the patient data in the database 122, and using the patient data, the patient monitor 102 generates medical intelligence for use in reporting and research. As described herein, the database 122 may include one or more non-relational databases, include a storage cluster, one or more relational databases and/or the like. The patient data is provided to the database 122, which is configured to parse, tag, and/or associate data elements for storage and analysis. The database 122 may include various modules, components, systems, infrastructures, and/or applications that may be combined in various ways, including into a single software application or multiple software applications. The database 122 is a distributed, scalable storage layer that is configured to store a large volume of structured and unstructured data. In one implementation, the database 122 replicates and distributes blocks of data through cluster nodes, along with numerous other features and advantages. As such, the database 122 generally manages the processing, storage, analysis, and retrieval of large volumes of data in a non-relational manner.

The database 122 serializes and stores the patient data, such that the medical intelligence may be generated based on a query. The database 122 processes a query in multiple parts at the cluster node level and aggregates the results to generate the medical intelligence In one implementation, the database 122 receives a query in structured query language (SQL), aggregates data stored in the database 122, and outputs the medical intelligence in a format enabling further management, analysis, and/or merging with other data sources. For example, the medical intelligence may be used to pre-populate: internal reports; external reports; reports to local, state, or national, registries; research, billing forms, statistical reports or forms, the patient's medical record; and/or the like. In one implementation, the patient data is captured using custom filters to study particular data points of emergency medical procedures, and the medical intelligence is used to pre-populate corresponding research forms. The database 122 may generate the medical intelligence using machine learning techniques, which generally refers to a machine learning through observing data that represents incomplete information about statistical happenings and generalizing such data to rules and/or algorithms that make predictions for future data, trends, and the like. Machine learning typically includes “classification” where machines learn to automatically recognize complex patterns and make intelligent predictions for a class.

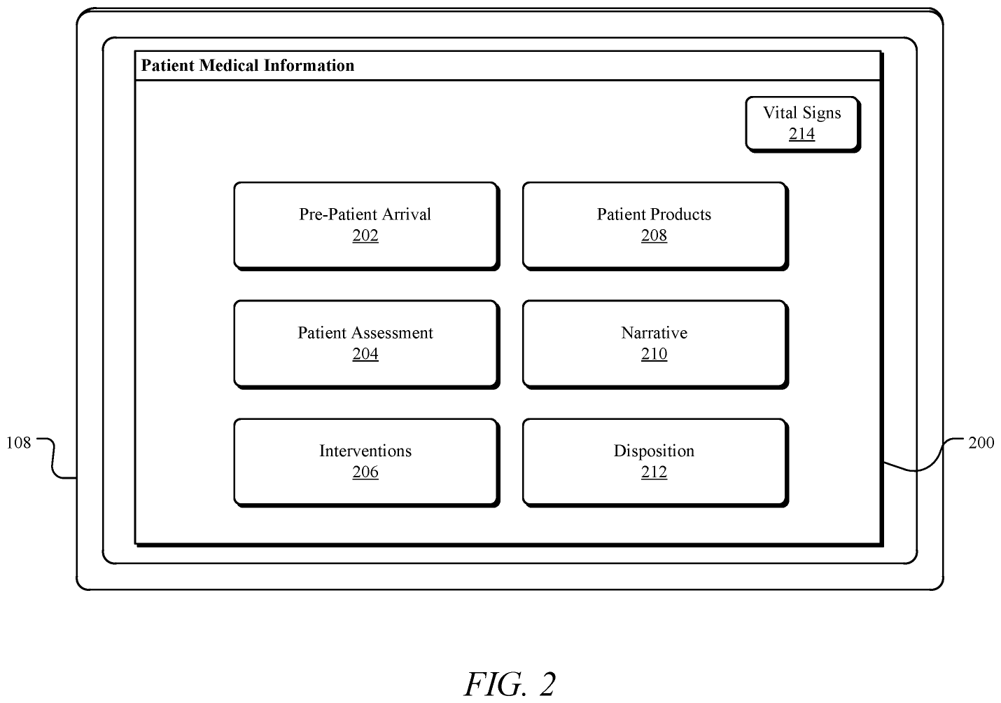

FIGS. 2-8 show example user interfaces 200-800 generated by the patient monitor 102 and displayed in a window of the scribe device 108 through which access to and interactions with the patient monitoring system 100, patient information, and other information are provided. In one implementation, the user interfaces 200-800 include trauma flow procedures for recording and monitoring patient information pertaining to treatment for an acute trauma, cardiac resuscitation, and/or similar emergency medical events. It will be appreciated by those skilled in the art that such depictions are exemplary only and not intended to be limiting.

Turning first to FIG. 2, a patient medical information user interface 200 provides a primary location for capturing and accessing patient information for different aspects of an emergency medical event. In one implementation, the patient medical information user interface 200 includes one or more emergency medical workflows (e.g., workflows 202-212). The workflows 202-212 facilitate quick and accurate capture of patient information following an emergency medical event, increasing situational awareness, information dissemination, real-time monitoring and communication, and the like during an emergency medical procedure for the patient. The workflows 202-212 further maximize resources and reduce error by pre-populating downstream records for the emergency medical procedure. The patient information captured with the workflows 202-212 additionally facilitate reporting for internal and external reports, registries, research, or statistical data, including billing capture. To quickly obtain timestamped vital signs of the patient during any point in the emergency medical procedure, a capture vital signs option 214 may be selected. The capture vital signs option 214 may be displayed on the patient medical information user interface 200 and/or with other user interfaces generated for the workflows 202-212 to facilitate vital sign monitoring.

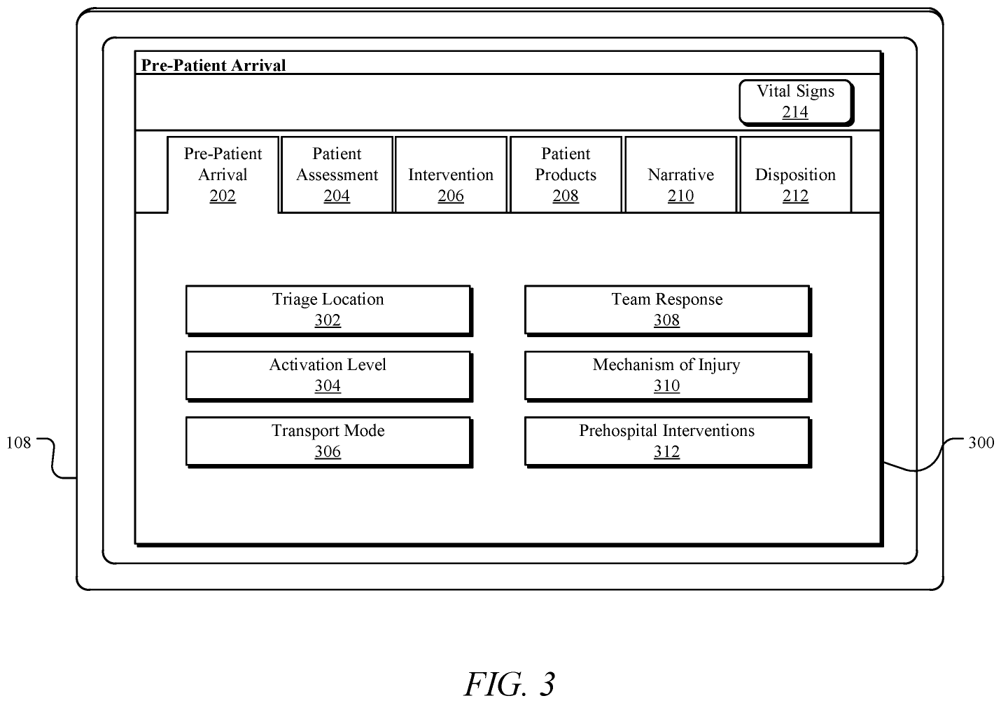

In one implementation, the emergency medical workflows 202-212 include a pre-patient arrival workflow 202, a patient assessment workflow 204, an interventions workflow 206, a patient products workflow 208, a narrative workflow 210, and a disposition workflow 212. Selection of one of the workflows 202-212 using the scribe device 108 causes information and/or one or more sub-workflows associated with the selected workflow to be presented on the scribe device 108. Selection of one of the workflows 202-212 may minimize the non-selected workflows to facilitate data input for the selected workflow, while providing quick access to the non-selected workflows, as needed. For example, as illustrated in FIGS. 3-8, the workflows 202-212 may be presented as tabs on each of the user interfaces 300-800 after selection of one of the workflows 202-212.

Referring to FIG. 3, in one implementation, selection of the pre-patient arrival workflow 202 causes a pre-patient arrival user interface 300 to be displayed with the scribe device 108. In one implementation, the pre-patient arrival user interface 300 includes a workflow for capturing data pertaining to a time prior to arrival of the patient at an emergency medical location, such as a hospital, trauma bay, or other location equipped for executing an emergency medical procedure for a patient following an emergency medical event. In one implementation, the pre-patient arrival user interface 300 is pre-populated from data captured using the remote device 114, the sensors 116, and/or other data sources. In another implementation, pre-patient arrival data is input into the pre-patient arrival user interface 300 using the scribe device 108.

In one implementation, the pre-patient arrival user interface 300 includes one or more sub-workflows for capturing pre-patient arrival data, including, without limitation, triage location 302, activation level 304, transport mode 306, team response 308, mechanism of injury 310, prehospital interventions 312, and the like. Selection of one of the sub-workflows 302-312 may open one or more windows, which may include customizable picklist(s), for capturing corresponding pre-patient arrival data. Some or all of the data may be timestamped, extracted, collated, and/or output for presentation on the display 118.

The triage location 302 may be used to capture pre-patient arrival data detailing a location at which a degree of urgency and/or order of treatment was assigned to a condition of the patient following an emergency medical event. The triage location 302 may present options for selection, including a scene of the emergency medical event, a transfer from another emergency medical facility, an emergency department (ED), and/or the like. In one implementation, a customizable picklist is presented listing local or frequent emergency medical facilities and emergency departments upon selection of the transfer and ED options, respectively. The activation level 304 details a level for use in triage, including, for example, level I, level II, level III, and consult. The level may be upgraded or downgraded using the pre-patient arrival user interface 300.

The transport mode 306 may be used to detail how the patient was transported to the emergency medical location. Options presented for selection in the transport mode 306 may include, without limitation, ambulance, helicopter, fixed wing, privately owned vehicle (POV), and/or the like. Selection of one of these options may present a customizable picklist for specifying an agency associated with the transport mode.

Many emergency medical locations enforce regulations specifying a time when emergency medical personnel must be present at the emergency medical location for response to an emergency medical event. For example, some regulations hold that personnel must be present within a particular time (e.g., 25 minutes) after being called or prior to arrival of the patient at the emergency medical location. The team response 308 thus details when each member of the emergency medical procedure team is called and arrives at the emergency medical location. The team members may include, without limitation, trauma surgeon, anesthesiologist, ED provider, primary ED registered nurse (RN), secondary ED RN, surgical intensive care unit (SICU) RN, operating room (OR) RN, ED resident, surgical resident, and other medical personnel. In one implementation, the time called and time arrived are manually input using the scribe device 108 for each of the team members. In another implementation, the time called and time arrived are received by the patient monitor 102 and pre-populated into the team response 308. For example, access cards for each team member utilizing radio frequency identification (RFID) or similar technology may be used to generate and communicate a timestamp for when the team member arrives at the emergency medical location.

The mechanism of injury 310 may be used to specify a cause, nature, and other details surrounding the emergency medical event. In one implementation, the mechanism of injury 310 provides categories of injury, including, but not limited to, fall/jump, penetrating, blunt, thermal, and other. The fall/jump category specifies a location a fall/jump occurred from, an approximate height of the location to a point of impact, and other information. The penetrating category specifies whether the emergency medical event was a gunshot wound (GSW) or other ballistic trauma, a stab wound, an impalement (including a type of wounding agent), and other penetration types. The blunt category specifies whether the injury was an assault, a crush injury, a pinned injury, a motor vehicle collision (MVC), or other blunt injury. The thermal category indicates whether the injury was caused by burn, flame, chemical, explosion, electrocution, cold exposure, or other thermal event. The other category specifies other mechanisms of injury, including, but not limited to, hanging, near drowning, machinery, animal (including type, bite, kicked, thrown, etc.), medical, found down, unresponsive, overdose, or other mechanism of injury.

Each of the categories may list one or more sub-categories that when selected may open a window for specifying details of the emergency medical event. For example, selecting MVC in the blunt category as the mechanism for injury may open a window presented on the scribe device 108 with additional options for detailing the mechanism of injury, including, without limitation, rollover, intrusion (e.g., in inches), starring wheel deformity, starred windshield, extended extrication (specifying time elapsed), vehicle type (e.g., car, truck, all-terrain vehicle, boat, motorcycle), position in vehicle (e.g., driver, passenger, front, rear), struck, thrown, ejected (e.g., in feet), rear-ended, T-bone (e.g., on driver side or passenger side), head-on, pedestrian struck, restraint devices (e.g., lap belt, shoulder belt, unrestrained, airbag deployed, helmet, protective clothing), and the like. The mechanism of injury 310 may further specify whether the emergency medical event was intention and/or work related.

The prehospital interventions 312 specify what interventions were taken in treating the patient prior to arrival at the emergency medical location. The prehospital interventions 312 thus provide information to prepare the emergency medical procedure team for structuring and executing the emergency medical procedure, including actions that need to be taken upon arrival of the patient. In one implementation, at least some of the prehospital interventions 312 is pre-populated with data captured using the remote device 114 and/or the sensors 116. The prehospital interventions 312 may include, without limitation, prehospital vital signs, airway interventions, breathing interventions, circulation interventions, tourniquet interventions, immobilization interventions, medications, cardiopulmonary resuscitation (CPR), and other interventions.

The prehospital vital signs captured may include blood pressure, heart rate, respiratory rate, oxygen saturation, Glasgow Coma Scale (GCS) (e.g., including eye, verbal, and motor responses), and the like. In one implementation, the prehospital vital signs are pre-populated with data captured using the remote device 114 and/or the sensors 116. Further, in one implementation, the prehospital vital signs are extracted and collated by the patient monitor 102 for presentation via the display 118.

The airway interventions specify any oxygen or intubated interventions for the patient prior to arrival at the emergency medical location. Selection of the oxygen intervention opens one or more windows for specifying the nature of the oxygen intervention, including, but not limited to, NC, NRB, BVM, OPA, NPA, and the like (e.g., in L/min). Similarly, selection of the intubated intervention opens one or more windows for specifying the nature of the intubated intervention, including, without limitation, surgical, oral, nasal, or alternate. Other windows may open upon selection of one of these options to further specify the nature of the interred intervention, including, for example, ETCO2, size, CM, LMA, King, Combi, or other, as applicable to the intubated intervention employed. In one implementation, pre-patient arrival data corresponding to the ETCO2 of the intubated intervention is extracted and collated for presentation via the display 118.

The breathing interventions correspond to any interventions for the patient to address breathing concerns prior to arrival at the emergency medical location, such as needle decompression, a chest tube, and/or the like. Selection of needle decompression may prompt the selection of additional details, such as a location of the needle decompression (e.g., left, right, anterior axillary, MCL, etc.). Similarly, selection of chest tube may prompt the selection of additional details, such as a location (e.g., left or right) and a size.

The circulation interventions specify any intravenous (IV) interventions, intraosseous (IO) interventions, and/or other circulation interventions utilized prior to the arrival of the patient at the emergency medical location. In one implementation, selection of the IV interventions opens a picklist for specifying a site of the IV intervention, and selection of the IO interventions opens a window for selecting a site (e.g., left, right, humerus, tibia). The circulation interventions may further include options for specifying whether crystalloids or blood interventions were provided to the patient prior to arrival at the emergency medical location and a corresponding quantity of each. In one implementation, the quantity is pre-populated with data captured using the remote device 114 and/or the sensors 116.

The tourniquet interventions include options for indicating whether a tourniquet was applied at an upper extremity and/or a lower extremity during prehospital procedures. Selection of either the upper or lower extremity may present options for indicating a side (e.g., left or right) and a time on and a time off. Similarly, immobilization interventions include options for specifying whether a C-Collar, backboard, scoop, splint, and/or the like were used prior to arrival at the emergency medical location. Selection of one of the immobilization interventions may prompt input of additional details, including, without limitation, upper extremity, lower extremity, right, left, and/or a picklist of customizable options defined based on local situations.

The medical interventions specifies any medications provided, for example, via rapid sequence induction (RSI) or other medications. Selection of RSI and/or other medications may open a picklist for specifying the specific medication and a dose. In one implementation, each medication intervention is timestamped to identify when the medication was provided to the patient. In one implementation, pre-patient arrival data corresponding to dose and timestamp of the medical intervention is extracted and collated for presentation via the display 118.

The CPR interventions detail whether any CPR procedures were undertaken. Upon selection, the patient monitor 102 prompts additional input, including a time of the CPR procedure and any medications used in the CPR procedure. Selection of a medication may require input of a dose and time provided.

Other prehospital interventions may include, without limitation, Foley, gastric tube, glucose, and/or the like. Selection of one of the other prehospital interventions may prompt input of additional detail. For example, selection of glucose may require specification of an amount (e.g., in mg/dl), and selection of Foley or gastric tube may require specification of a size.

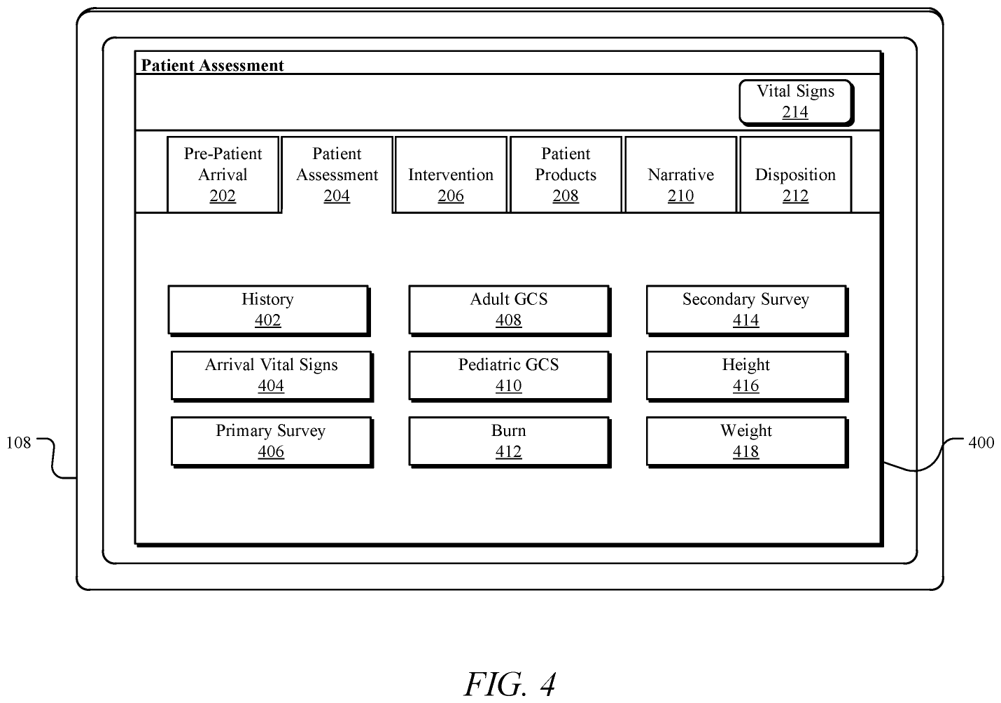

As can be understood from FIG. 4, in one implementation, selection of the patient assessment workflow 204 causes a patient assessment user interface 400 to be displayed with the scribe device 108. In one implementation, the patient assessment user interface 400 includes a workflow for capturing data pertaining to an assessment of the patient after arrival at the emergency medical location. In one implementation, the patient assessment user interface 400 is pre-populated from data captured using the sensors 116 and/or other data sources. In another implementation, patient assessment data is input into the patient assessment user interface 400 using the scribe device 108. The patient assessment data captured using the patient assessment user interface 400 may automatically pre-populate a physician history and physical form for review and approval by the physician regarding the emergency medical procedure.

In one implementation, the patient assessment user interface 400 includes one or more sub-workflows for capturing patient assessment data, including, without limitation, history 402, arrival vital signs 404, primary survey 406, adult GCS, pediatric CGS, burn 412, secondary survey 414, and the like. In one implementation, height 416 and weight 418 are separate sub-workflows. In another implementation, a height and weight of the patient are entered using the history 402. Selection of one of the sub-workflows 402-418 may open one or more windows, which may include customizable picklist(s), for capturing corresponding patient assessment data. Some or all of the data may be timestamped, extracted, collated, and/or output for presentation on the display 118.

The history 402 provides options for detailing a history of the patient, including, but not limited to, allergies, medications, past illnesses, pregnancies, last meal, events, tetanus immunization, height, weight, body mass index (BMI), and/or the like. In one implementation, the past illnesses and pregnancies may be selected from a picklist, and input of other details corresponding to the events and tetanus immunization (e.g., date administered) may be prompted. The BMI may be automatically calculated based on the height and weight.

The arrival vital signs 404 captured may include temperature, blood pressure, heart rate, respiratory rate, oxygen saturation, GCS (e.g., including eye, verbal, and motor responses), and the like. In one implementation, the patient monitor 102 indicates whether the arrival vital signs 404 were automatically or manually captured and includes a timestamp indicating when the arrival vital signs 404 were captured. The arrival vital signs 404 may be extracted and collated by the patient monitor 102 for presentation via the display 118.

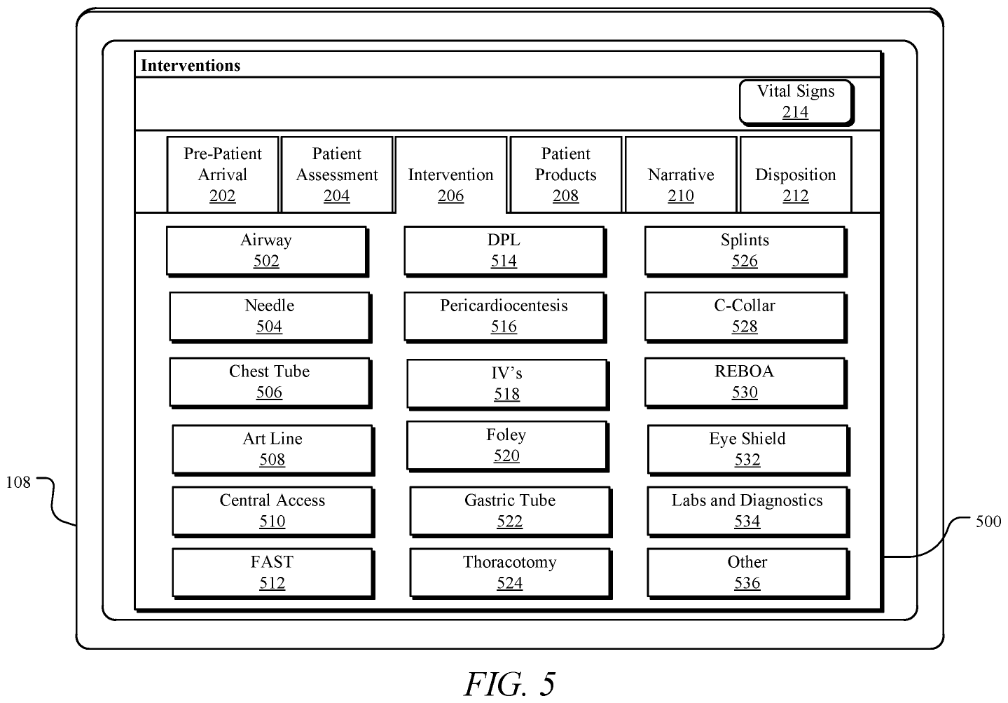

The primary survey 406 includes a workflow for ensuring that the most life threatening situations are addressed first. The primary survey 406 prompts examination and corresponding documentation of airway, breathing, circulation, disability, and exposure/environment. In one implementation, following examination of the airway, options for documenting the results of the examination are presented, including, but not limited to, patent, clear, obstructed, assisted, ET-PTA, oral, nasal, c-spine immobilized, and/or the like. Following examination of the patient breathing, options for documenting the results of the examination are presented, including, but not limited to, spontaneous, unlabored, labored, agonal, absent, breath sounds, chest symmetry, and/or the like. Selection of breath sounds may prompt additional input, such as a side (e.g., right or left) and nature (e.g., clear, rales, wheezes, diminished, absent). Also, selection of chest symmetry may prompt additional input, such as symmetrical, asymmetrical, or paradoxical. In one implementation, following examination of the circulation, options for documenting the results of the examination are presented, including, but not limited to, skin (e.g., warm/dry/pink, cool, pale, cyanotic, diaphoretic, etc.) and capillary refill (e.g., less than three seconds or greater than three seconds). Patient assessment data detailing the results of disability examination may include, the adult GCS 408, the pediatric GCS 410, and/or pupil dilation (e.g., 2-9 m). The exposure examination documentation indicates a use of warm blankets, warm fluids, warming lights, bair hugger, cooling blankets, and/or the like. Selection of interventions corresponding to any of the examinations may redirect to an interventions user interface 500, described with respect to FIG. 5, to specify the nature of the intervention.

The burn section 412 includes patient assessment data for caring for and documenting any burns to the patient. In one implementation, selection of the burn section 412 presents a burn sheet with images enabling tabulation of a percentage burn estimate and burn resources, including, without limitation the Parkland formula, which is a burn formula used to estimate the amount of replacement fluid required for the first 24 hours in a burn patient so as to ensure they remain hemodynamically stable.

The secondary survey 414 includes a workflow for detailing additional patient assessment data, including a trauma site and nature, a mental status, face/head status, pupil reaction status, ears status, neck status, chest status, breathing sounds, abdomen status, pelvis status, upper extremities status, lower extremities status, and back status. In one implementation, the trauma sites are illustrated using a diagram.

Turning to FIG. 5, in one implementation, selection of the interventions workflow 206 causes a interventions user interface 500 to be displayed with the scribe device 108. In one implementation, the interventions user interface 500 includes a workflow for recording and monitoring any medical interventions or other inputs to the patient during an emergency medical procedure at the emergency medical location. In one implementation, the interventions user interface 500 is automatically populated and updated from data captured using the sensors 116 and/or other data sources. In another implementation, patient interventions data is input into the interventions user interface 500 using the scribe device 108.

In one implementation, the interventions user interface 500 includes one or more sub-workflows for capturing patient intervention data, including, without limitation, airway 502, breathing (e.g., needle decompression 504 and chest tube 506), circulation (e.g., art line 508 or central access 510), focused assessment with sonography for trauma (FAST) 512 (including extended FAST (eFAST)), Diagnostic peritoneal lavage (DPL) 514, pericardiocentesis 516, IV's 518, Foley 520, gastric tube 522, thoracotomy 524, splints 526, C-Collar 528, Resuscitative Endovascular Balloon Occlusion of the Aorta (REBOA) 530, eye shield 532, labs and diagnostics 534, and other interventions 536. Selection of one of the sub-workflows 502-536 may open one or more windows, which may include customizable picklist(s) or other options for capturing corresponding patient intervention data. Some or all of the data may be timestamped, extracted, collated, and/or output for presentation on the display 118. The sub-workflows 502-536 may each prompt input detailing the medical personnel who performed the intervention.

Selection of the airway interventions 502, such as ETT or Cric, prompts input regarding whether the airway intervention was oral or nasal, which further prompts input detailing BBS present, EtCO2 change, cm teeth/nare, selection of an adult size or a pediatric size, and/or the like. Selection of needle decompression 504 may prompt the selection of additional details, such as a location of the needle decompression (e.g., left, right, anterior, axillary, etc.). Similarly, selection of chest tube 506 may prompt the selection of additional details, such as a location (e.g., left or right) and a size. Cubic centimeters (cc) of blood may further be specified for the needle decompression 504 and/or the chest tube 506.

In one implementation, selection of the art line 508 requires input regarding a location (e.g., right or left), and selection of the central access 510 requires input regarding a location (e.g., right or left), whether the central access was subclavian, U, or femoral, a type, and the like. The FAST 512 and eFAST may each prompt input regarding whether the intervention was positive, negative, or indeterminate. The FAST 512 may further prompt information regarding whether the intervention was pericardial, RUQ, LUQ, suprapubic, and/or the like, and the eFAST may further detail right or left. The DPL 514 and the pericardiocentesis 516 each similarly specifies positive or negative. The IV's 518, upon selection, detail each of the IV's used and a corresponding gauge and site for each. Selection of the Foley 520 requires specification of a size. The gastric tube 522 prompts input regarding a site (e.g., OG, NG, R, L) and a size. The thoracotomy 524 requires specification of when a cross clamp was on and when a cross clamp was off.

Selection of the splints 526 requires input regarding a location and pulses (e.g., palpable, dopplerable, absent). The C-Collar 528 includes corresponding data indicating a time applied and a time removed. Similarly, other immobilization interventions may be included in the other interventions 536, such as a backboard and pelvic binder, which may each require additional input regarding a time applied and a time discontinued. The eye shield 532 specifies a site (e.g., right, left, or both).

The labs and diagnostics 534 details what labs were ordered and when those labs were ordered and drawn. Further, the labs and diagnostics 534 details if and when an X-ray, CT, or other diagnostic tool or imaging were ordered and performed, along with a location (e.g., chest, pelvis, extremity, head, neck, abdomen, panscan, or other). The labs and diagnostics 534 thus inform the emergency medical personnel what to expect and when during the emergency medical procedure. The other interventions 536 may include additional interventions, customized for particular situations or environments. For example, the other interventions 536 may include hemorrhage control methods for use in a military or other tactical environment, with options for specifying the hemorrhage control method used (e.g., Celox, ChiloFlex, comb. gauze, direct pressure, field dressing, helmon, quikclot, none) and a time used.

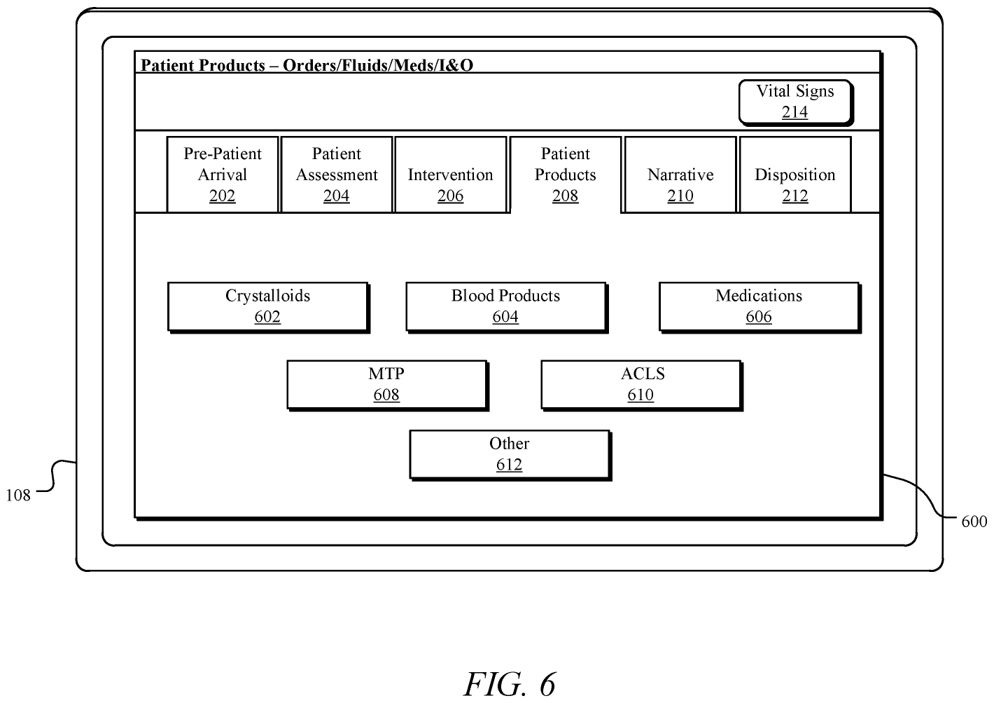

As can be understood from FIG. 6, selection of the patient products workflow 208 causes a patient products user interface 600 to be displayed with the scribe device 108. In one implementation, the patient products user interface 600 includes a workflow for recording and monitoring any orders, fluids, blood products, medications, or other products provided to the patient during the emergency medical procedure. In one implementation, the patient products user interface 600 is automatically populated and updated from data captured using the sensors 116 and/or other data sources. In another implementation, patient products data is input into the patient products user interface 600 using the scribe device 108.

In one implementation, the patient products user interface 600 includes one or more sub-workflows for capturing patient products data, including, without limitation, crystalloids 602, blood products 604, medications 606, massive transfusion protocol (MTP) 608, Advanced Cardiac Life Support (ACLS) 610, and other products 612. Selection of one of the sub-workflows 602-612 may open one or more windows, which may include customizable picklist(s) or other options for capturing corresponding patient products data. Some or all of the data may be timestamped, extracted, collated, and/or output for presentation on the display 118.

The crystalloids 602 are product comprising aqueous solutions of mineral salts or other water-soluble molecules that may be provided as an intravenous therapy. Where the patient is given crystalloids, the crystalloids 602 specifies NS or LR and a time the liter completes. In one implementation, the crystalloids 602 provided are automatically monitored and additive.

The blood products 604 specifies which blood products were provided to the patient, the input, the number of units, and when. Such blood products may include, without limitation, packed cells, FFP, platelets, cryo, and the like. Selection of the MTP 608 may display patient products data corresponding to an MTP. In one implementation, patient product data corresponding to the MTP 608 is recorded and monitored separately and in parallel to the other patient product data, for example, using the MTP device 112. The MTP 608 may specify the time, RBC, FFP, platelets, and cryo.

The medications 606 detail the medications provided to the patient, including a time and dosage. The medications 606 may include, without limitation, RSI, analgesia, sedatives/anesthetics, antibiotics, paralytics, pressors, cardiac, and/or the like. The other patient products 612 may include, without limitation, HTS, manitol, and/or the like with a corresponding amount specified using, for example, a picklist.

The ACLS 610 provides various workflows corresponding to cardiac life support, including, but not limited to, cardiac arrest algorithm, cardiac arrest circular algorithm, immediate post-cardiac arrest care algorithm, tachycardia with a pulse algorithm, bradycardia with a pulse algorithm, acute coronary syndromes algorithm, and suspected stoke algorithm.

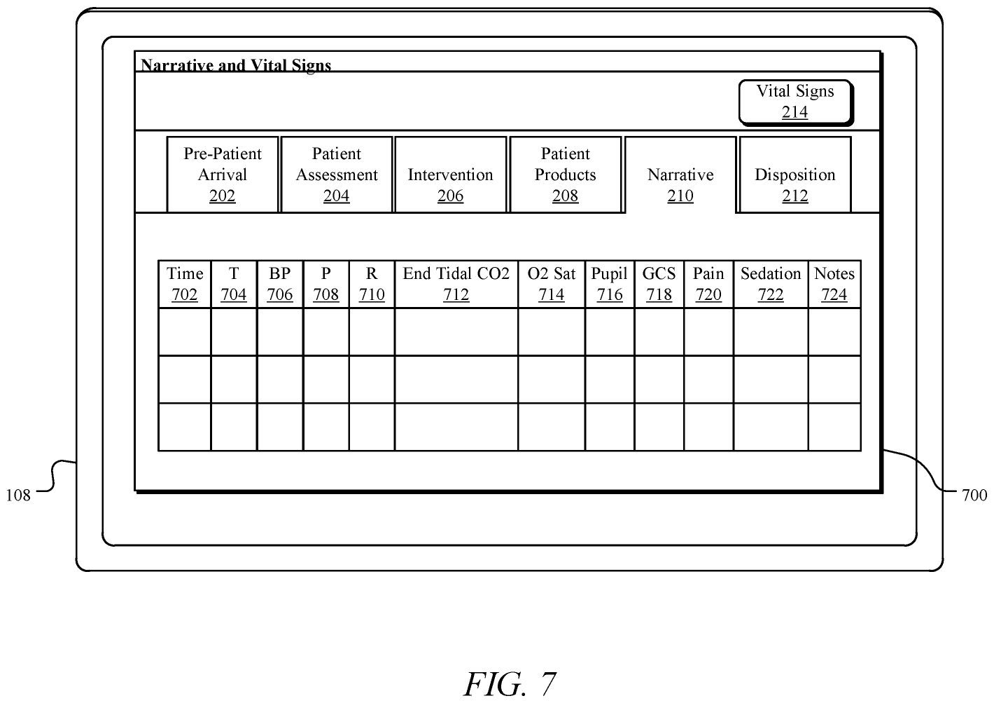

Referring to FIG. 7, selection of the narrative workflow 210 causes a narrative user interface 700 to be displayed with the scribe device 108. In one implementation, the narrative user interface 700 provides a table for capturing and monitoring vital signs and other critical information for the patient and recording associated narratives during the emergency medical procedure. The narrative data displayed with the narrative user interface 700 may include, without limitation, time 702, temperature 704, blood pressure 706, pulse 708, respiratory rate 710, end of tidal carbon dioxide 712, oxygen saturation 714, pupil responsiveness 716, GCS 718, pain 720 (e.g., on a scale of 1-10), sedation 722 (e.g., on a scale of 1-7), and notes 724. In one implementation, the narrative user interface 700 is automatically populated and updated from data captured using the sensors 116 and/or other data sources. In another implementation, patient products data is input into the narrative user interface 700 using the scribe device 108. Some or all of the data may be timestamped, extracted, collated, and/or output for presentation on the display 118.

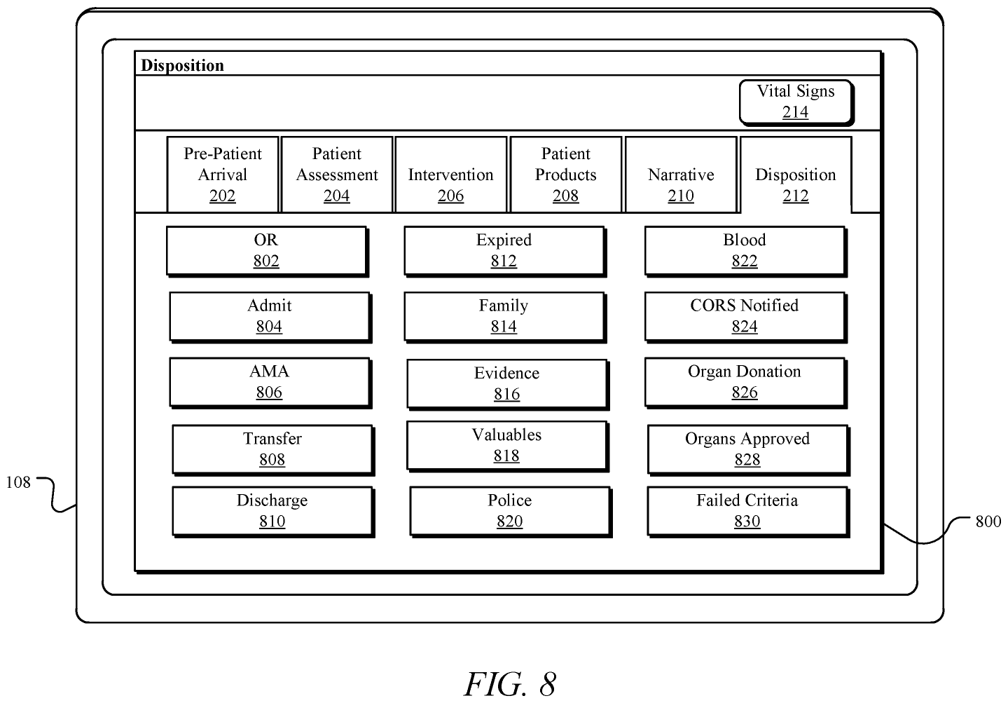

Selection of the disposition workflow 212 causes a disposition user interface 800 to be displayed with the scribe device 108, as shown in FIG. 8. In one implementation, the disposition user interface 800 includes a workflow for recording dispositions of various aspects of the emergency medical procedure and/or event. In one implementation, the disposition user interface 800 is automatically populated and updated from data captured using the sensors 116, the remote device 114, and/or other data sources. In another implementation, patient disposition data is input into the disposition user interface 800 using the scribe device 108.

In one implementation, the disposition user interface 800 includes one or more sub-workflows for capturing patient disposition data, including, but not limited to, operating room (OR) 802, admit 804, AMA 806, transfer 808, discharge 810, expired 812, family 814, evidence 816, valuables 818, police 820, blood 822, CORS notified 824, organ donation 826, organs approved 828, failed criteria 830, and/or the like. Selection of one of the sub-workflows 802-830 may open one or more windows, which may include customizable picklist(s) or other options for capturing corresponding patient disposition data. Some or all of the data may be timestamped, extracted, collated, and/or output for presentation on the display 118.

The OR 802 disposition indicates whether to report to the RN or anesthesia, and the admit 804 disposition specifies a bed and report called to intensive care unit (ICU) or floor. The AMA 806 disposition details whether the form was signed, signing the form was refused, or the patient was unable to be located. The transfer 808 disposition details where to transfer the patient (e.g., selected from a picklist), whether the form was complete, and whether valuables, copy and record are to be sent with the patient. The discharge 810 disposition specifies whether the patient is being discharged home and whether the patient is accompanied and by whom. The expired 812 disposition details whether the ME is notified and pronounced by whom.

The family 814 disposition details whether the family visited, was called, or whether the family could not be located. The evidence 816 specifies whether the evidence, including clothing, was released to the patient, police, security, and/or the like. The valuables 818 details whether the patient's valuables are with the patient, security, family, or another party. The police 820 details the department involved, as well as an officer name and badge number. The blood 822 indicates whether the blood was sent to the OR, ICU, or returned to the blood bank.

The CORS notified 824 section details whether an organ recovery service (e.g., Colorado) was notified, by whom, when, and provides a signature. The organ donation 826 indicates whether the patient is a donor, and the organs approved 828 details which organs (e.g., eye, bone, tissue, other) are approved for donation. The failed criteria 830 specifies which criteria is failed.

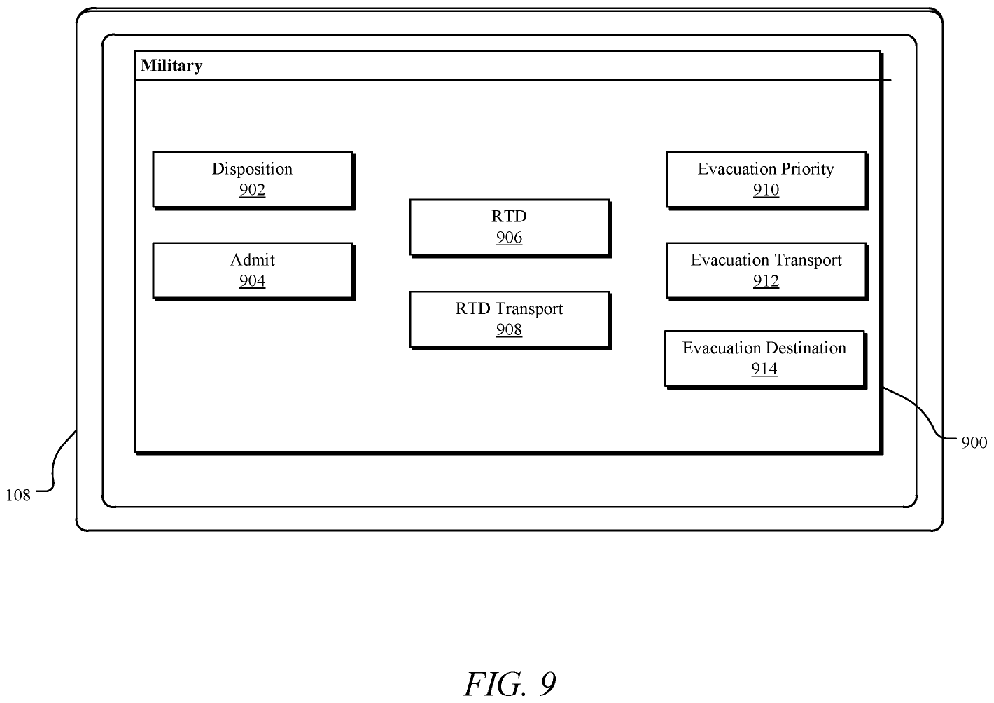

FIG. 9 shows an example military trauma user interface 900 generated by the patient monitor 102 and displayed in a window of the user device 104, such as the scribe device 108, through which access to and interactions with the patient monitoring system 100, patient information, and other information are provided. In one implementation, the military trauma user interface 900 includes trauma flow procedures for recording and monitoring patient information pertaining to treatment for an acute trauma, cardiac resuscitation, and/or similar emergency medical events in a military or tactical environment. It will be appreciated by those skilled in the art that such depictions are exemplary only and not intended to be limiting.

In one implementation, the military trauma user interface 900 includes one or more sub-workflows for capturing patient data in a military environment, including, but not limited to, disposition 902, admit 904, RTD 906, RTD transport 908, evacuation priority 910, evacuation transport 912, and evacuation destination 914, and/or the like. Selection of one of the sub-workflows 902-912 may open one or more windows, which may include customizable picklist(s) or other options for capturing corresponding patient data. Some or all of the data may be timestamped, extracted, collated, and/or output for presentation on the display 118 or other user device 104.

The disposition 902 details a date and time and condition of the patient, and the admit 904 indicates whether to admit the patient to the OR, ICU, ICW, or the like. The RTD 906 specifies full, quarters, or profile, and an RTD unit. The RTD transport 908 details a mode of transport, such as ambulatory, W/C, or the like. The evacuation priority 910 defines a priority for evacuating the patient, such as routine, priority, or urgent, and the evacuation transport 912 specifies whether the patient will be evacuated using MEDEVAC (e.g., rotary wing, fixed wing, medtech, AE, CCATT, critical are, etc.), ground (e.g., medical or non-medical), and a mode of transport (e.g., ambulatory, litter, W/C, vacuum spine board, etc.). The evacuation destination 914 details where to evacuate the patient, including, but not limited to, a host nation, CASF, and a specified facility.

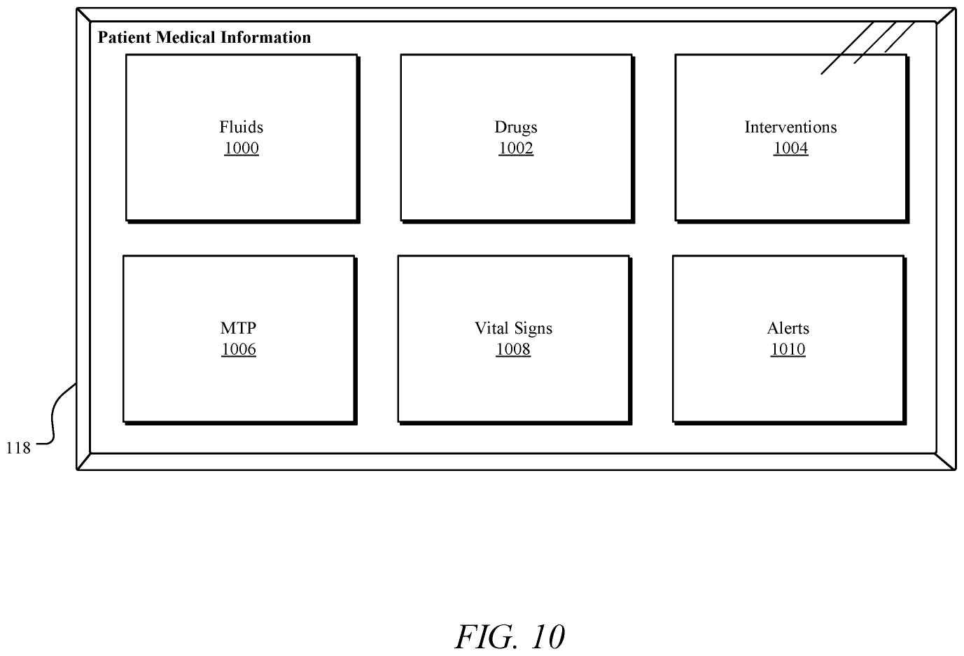

As detailed herein, patient information, particularly critical patient information, may be extracted, collated, and output for presentation on the display 118 during an emergency medical procedure to facilitate accurate data capture, situational awareness, and communication among emergency medical team members. Referring to FIG. 10, in one implementation, the patient information includes, without limitation, fluids 1000, drugs 1002, interventions 1004, MTP 1006 (if applicable), vital signs 1008, and alerts 1010.

The fluids 1000 and drugs 1002 includes patient information extracted and collated from the patient products data captured, for example using the patient products user interface 600, and the interventions 1004 includes patient information extracted and collated from the patient interventions data captured, for example using the interventions user interface 500. In one implementation, the MTP 1006 is presented on the display 118 only where a MTP is administered to the patient during the emergency medical procedure to monitor status. The MTP 1006 may include patient information extracted and collated from data captured using the MTP device 112 and other sources. The vital signs 1108 provides a quick reference of the vital signs of the patient, including, but not limited to, temperature, blood pressure, heart rate, respiratory rate, oxygen saturation, GCS, and the like.

As detailed herein, alerts may be visual, audio, and/or haptic, and output via various computing devices, including the user devices 104, the display 118, and/or the like. In one implementation, the alerts are presented on the display 118 in the alerts 1010. The alerts 1010 may include, without limitation, airway/breathing alerts, circulation/shock alerts, disability alerts, and miscellaneous alerts. Additionally, there may be alerts customized for pediatrics or other particular conditions or situations applicable to the patient.

The alerts are generated based on patient information extracted and collated from the patient data and based on one or more thresholds or other rules. In one implementation, the airway/breathing alerts include, end-tidal carbon dioxide, oxygen saturation, GCS, RR, and/or the like. As a particular example, an airway/breathing alert may be generated when: oxygen saturation is less than 91% (slow) or less than 85% (fast); GCS is 9 (slow) or is 8 or greater (fast); and/or RR is greater than 20 (slow) or greater than 30 (fast).

In one implementation, the circulation alerts include, crystalloid infusion, heat rate, blood infusion, blood sent to lab, blood type complete, cross complete, FFP ready, MTP in progress, ABG available, chest tube output, blood pressure, tourniquet time, and/or the like. As a particular example, a circulation alert may be generated when: crystalloid infusion is 1.5 liters (slow) or greater than 2 liters (fast); heart rate is less than 40 or greater than 130; blood infusion is greater than 6 units in 60-90 minutes; ABG is available when oxygen (PaO2) is less than 60 (slow) or less than 50 (fast), as well as when carbon dioxide is greater than 50 (slow) or greater than 60 (fast) or less than 30 (slow); the chest tube output is greater than 1500 cc at initial tube placement (fast) or greater than 500 cc/hr (fast); the systolic blood pressure is less than 100 (slow) or less than 90 (fast); and/or after the elapsed time for the tourniquet.

In one implementation, the disability alerts include, dilated pupil(s), not moving extremities, and/or the like. As a particular example, a disability alert may be generated when one or more of the pupils is greater than 5 mm. The miscellaneous alerts include, base deficit, PCO2, lactate, platelets, Hct, Hb, PT/INR, TEG, temperature, scrotal hematoma, urethral blood, and/or the like. As a particular example, a miscellaneous alert may be generated when: the base deficit is more than −7 (slow) or more than (−10) fast); the PCO2 is more than 45 (slow); the platelets are less than 50 (slow); the Hct is less than 20% (fast); the Hb is less than 7 gm/dl (fast); the PT/INR is greater than 1.5 (slow) or 2.0 (fast); and/or the like.

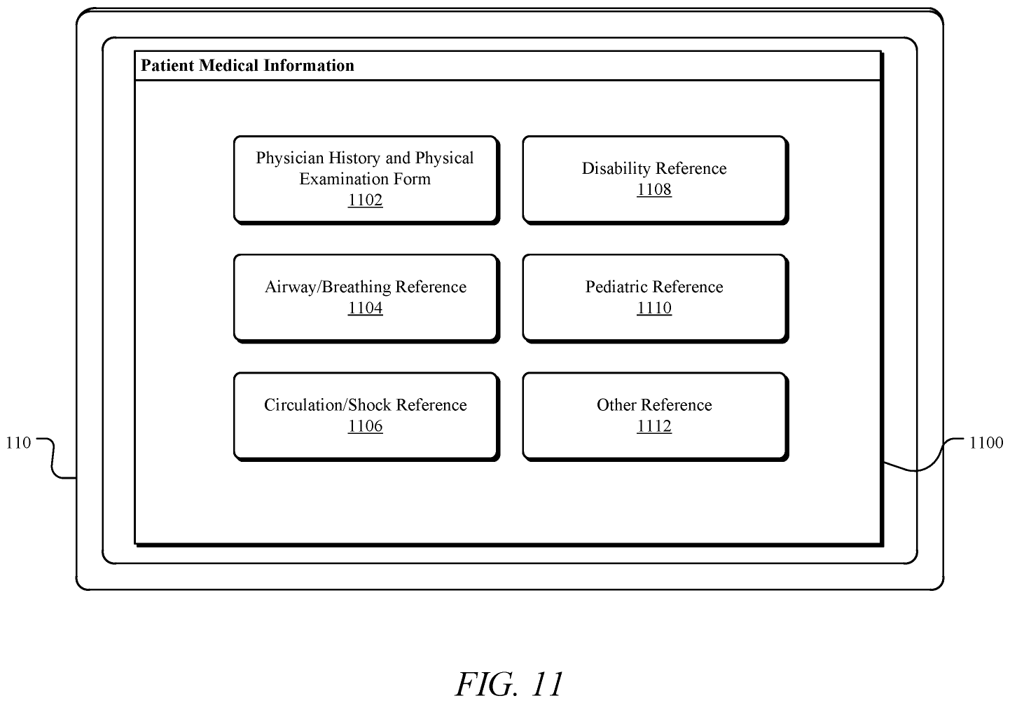

FIG. 11 shows an example patient medical user interface 1100 generated by the patient monitor 102 and displayed in a window of the physician device 110 through which access to and interactions with the patient monitoring system 100, patient information, and other information are provided. In one implementation, the patient medical user interface 1100 includes trauma flow procedures for recording patient information and accessing reference information during an acute trauma, cardiac resuscitation, and/or similar emergency medical events. It will be appreciated by those skilled in the art that such depictions are exemplary only and not intended to be limiting.

In one implementation, the patient medical user interface 1100 includes a physician history and physical examination form 1102 for review, revision, and approval by the physician. The physician history and physical examination form 1102 may be pre-populated with data captured using and extracted from the user interfaces 200-900. For example, the patient assessment data captured using the patient assessment user interface 400 may automatically pre-populate a physician history and physical form for review and approval by the physician regarding the emergency medical procedure.

The patient medical user interface 1100 may further includes various references providing quick access to information that may be useful to the physician during the emergency medical procedure. In one implementation, the references include, without limitation, airway/breathing references 1104, circulation/shock references 1106, disability references 1108, pediatric references 1110, and other references 1112. The airway/breathing references 1104 may include, for example, the LEMON assessment, Mallampati Classification, RSI drug list detailing the benefits and downsides, and/or the like. The circulation/shock references 1106 may include, for example, Pediatric Pearls, MTP protocol, interaction of interosseous device instructions, shock classifications, tourniquet time, and/or the like. The disability references 1108 may include, for example, hypertonic solution infusion, dermatome and myotome charts, and/or the like. The pediatric references 1110 may include, for example, Broselow Color, heart rate, pediatric GCS, and/or the like. The other references 1112 may include, for example, TEG, burn chart and table, Parkland formula, chemical burns, electrical burns, pregnancy, transfer form, ocular trauma, tourniquet time, and/or the like.

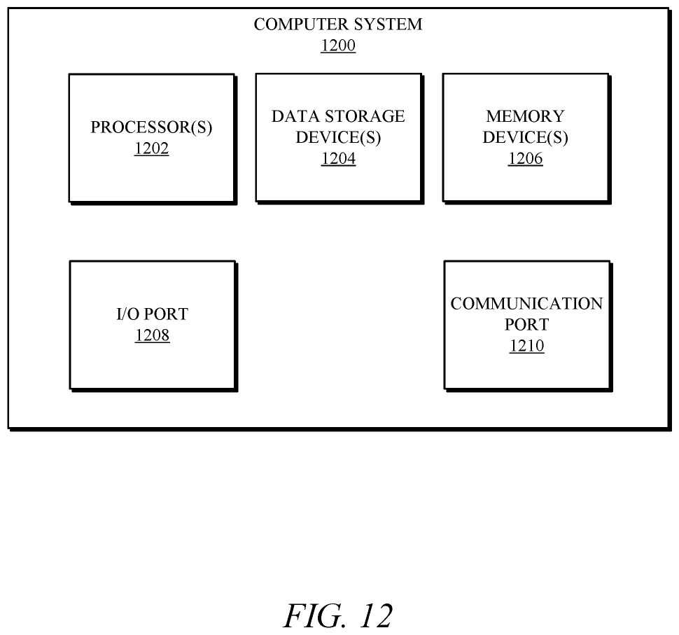

Referring to FIG. 12, a detailed description of an example computing system 1200 having one or more computing units that may implement various systems and methods discussed herein is provided. The computing system 1200 may be applicable to the user devices 104, the server 120, the display 118, and other computing or network devices. It will be appreciated that specific implementations of these devices may be of differing possible specific computing architectures not all of which are specifically discussed herein but will be understood by those of ordinary skill in the art.

The computer system 1200 may be a computing system is capable of executing a computer program product to execute a computer process. Data and program files may be input to the computer system 1200, which reads the files and executes the programs therein. Some of the elements of the computer system 1200 are shown in FIG. 12, including one or more hardware processors 1202, one or more data storage devices 1204, one or more memory devices 1208, and/or one or more ports 1208-1210. Additionally, other elements that will be recognized by those skilled in the art may be included in the computing system 1200 but are not explicitly depicted in FIG. 12 or discussed further herein. Various elements of the computer system 1200 may communicate with one another by way of one or more communication buses, point-to-point communication paths, or other communication means not explicitly depicted in FIG. 12.

The processor 1202 may include, for example, a central processing unit (CPU), a microprocessor, a microcontroller, a digital signal processor (DSP), and/or one or more internal levels of cache. There may be one or more processors 1202, such that the processor 1202 comprises a single central-processing unit, or a plurality of processing units capable of executing instructions and performing operations in parallel with each other, commonly referred to as a parallel processing environment.

The computer system 1200 may be a conventional computer, a distributed computer, or any other type of computer, such as one or more external computers made available via a cloud computing architecture. The presently described technology is optionally implemented in software stored on the data stored device(s) 1204, stored on the memory device(s) 1206, and/or communicated via one or more of the ports 1208-1210, thereby transforming the computer system 1200 in FIG. 12 to a special purpose machine for implementing the operations described herein. Examples of the computer system 1200 include personal computers, terminals, workstations, mobile phones, tablets, laptops, personal computers, multimedia consoles, gaming consoles, set top boxes, and the like.

The one or more data storage devices 1204 may include any non-volatile data storage device capable of storing data generated or employed within the computing system 1200, such as computer executable instructions for performing a computer process, which may include instructions of both application programs and an operating system (OS) that manages the various components of the computing system 1200. The data storage devices 1204 may include, without limitation, magnetic disk drives, optical disk drives, solid state drives (SSDs), flash drives, and the like. The data storage devices 1204 may include removable data storage media, non-removable data storage media, and/or external storage devices made available via a wired or wireless network architecture with such computer program products, including one or more database management products, web server products, application server products, and/or other additional software components. Examples of removable data storage media include Compact Disc Read-Only Memory (CD-ROM), Digital Versatile Disc Read-Only Memory (DVD-ROM), magneto-optical disks, flash drives, and the like. Examples of non-removable data storage media include internal magnetic hard disks, SSDs, and the like. The one or more memory devices 1206 may include volatile memory (e.g., dynamic random access memory (DRAM), static random access memory (SRAM), etc.) and/or non-volatile memory (e.g., read-only memory (ROM), flash memory, etc.).

Computer program products containing mechanisms to effectuate the systems and methods in accordance with the presently described technology may reside in the data storage devices 1204 and/or the memory devices 1206, which may be referred to as machine-readable media. It will be appreciated that machine-readable media may include any tangible non-transitory medium that is capable of storing or encoding instructions to perform any one or more of the operations of the present disclosure for execution by a machine or that is capable of storing or encoding data structures and/or modules utilized by or associated with such instructions. Machine-readable media may include a single medium or multiple media (e.g., a centralized or distributed database, and/or associated caches and servers) that store the one or more executable instructions or data structures.

In some implementations, the computer system 1200 includes one or more ports, such as an input/output (I/O) port 1208 and a communication port 1210, for communicating with other computing, network, or vehicle devices. It will be appreciated that the ports 1208-1210 may be combined or separate and that more or fewer ports may be included in the computer system 1200.

The I/O port 1208 may be connected to an I/O device, or other device, by which information is input to or output from the computing system 1200. Such I/O devices may include, without limitation, one or more input devices, output devices, and/or environment transducer devices.

In one implementation, the input devices convert a human-generated signal, such as, human voice, physical movement, physical touch or pressure, and/or the like, into electrical signals as input data into the computing system 1200 via the I/O port 1208. Similarly, the output devices may convert electrical signals received from computing system 1200 via the I/O port 1208 into signals that may be sensed as output by a human, such as sound, light, and/or touch. The input device may be an alphanumeric input device, including alphanumeric and other keys for communicating information and/or command selections to the processor 1202 via the I/O port 1208. The input device may be another type of user input device including, but not limited to: direction and selection control devices, such as a mouse, a trackball, cursor direction keys, a joystick, and/or a wheel; one or more sensors, such as a camera, a microphone, a positional sensor, an orientation sensor, a gravitational sensor, an inertial sensor, and/or an accelerometer; and/or a touch-sensitive display screen (“touchscreen”). The output devices may include, without limitation, a display, a touchscreen, a speaker, a tactile and/or haptic output device, and/or the like. In some implementations, the input device and the output device may be the same device, for example, in the case of a touchscreen.

The environment transducer devices convert one form of energy or signal into another for input into or output from the computing system 1200 via the I/O port 1208. For example, an electrical signal generated within the computing system 1200 may be converted to another type of signal, and/or vice-versa. In one implementation, the environment transducer devices sense characteristics or aspects of an environment local to or remote from the computing device 1200, such as, light, sound, temperature, pressure, magnetic field, electric field, chemical properties, physical movement, orientation, acceleration, gravity, and/or the like. Further, the environment transducer devices may generate signals to impose some effect on the environment either local to or remote from the example computing device 1200, such as, physical movement of some object (e.g., a mechanical actuator), heating or cooling of a substance, adding a chemical substance, and/or the like.

In one implementation, a communication port 1210 is connected to a network by way of which the computer system 1200 may receive network data useful in executing the methods and systems set out herein as well as transmitting information and network configuration changes determined thereby. Stated differently, the communication port 1210 connects the computer system 1200 to one or more communication interface devices configured to transmit and/or receive information between the computing system 1200 and other devices by way of one or more wired or wireless communication networks or connections. Examples of such networks or connections include, without limitation, Universal Serial Bus (USB), Ethernet, Wi-Fi, Bluetooth®, Near Field Communication (NFC), Long-Term Evolution (LTE), and so on. One or more such communication interface devices may be utilized via the communication port 1210 to communicate one or more other machines, either directly over a point-to-point communication path, over a wide area network (WAN) (e.g., the Internet), over a local area network (LAN), over a cellular (e.g., third generation (3G) or fourth generation (4G)) network, or over another communication means. Further, the communication port 1210 may communicate with an antenna or other link for electromagnetic signal transmission and/or reception.

In an example implementation, the patient monitor 102, patient medical information, a plurality of internal and external databases, source databases, cached data on servers, and/or software and other modules and services may be embodied by instructions stored on the data storage devices 1204 and/or the memory devices 1206 and executed by the processor 1202. Patient monitoring and recording software and other modules and services may be embodied by instructions stored on such storage systems and executed by the processor 1202.

The system set forth in FIG. 12 is but one possible example of a computer system that may employ or be configured in accordance with aspects of the present disclosure. It will be appreciated that other non-transitory tangible computer-readable storage media storing computer-executable instructions for implementing the presently disclosed technology on a computing system may be utilized.

In the present disclosure, the methods disclosed may be implemented as sets of instructions or software readable by a device. Further, it is understood that the specific order or hierarchy of steps in the methods disclosed are instances of example approaches. Based upon design preferences, it is understood that the specific order or hierarchy of steps in the method can be rearranged while remaining within the disclosed subject matter. The accompanying method claims present elements of the various steps in a sample order, and are not necessarily meant to be limited to the specific order or hierarchy presented.

The described disclosure may be provided as a computer program product, or software, that may include a non-transitory machine-readable medium having stored thereon instructions, which may be used to program a computer system (or other electronic devices) to perform a process according to the present disclosure. A machine-readable medium includes any mechanism for storing information in a form (e.g., software, processing application) readable by a machine (e.g., a computer). The machine-readable medium may include, but is not limited to, magnetic storage medium, optical storage medium; magneto-optical storage medium, read only memory (ROM); random access memory (RAM); erasable programmable memory (e.g., EPROM and EEPROM); flash memory; or other types of medium suitable for storing electronic instructions.

While the present disclosure has been described with reference to various implementations, it will be understood that these implementations are illustrative and that the scope of the present disclosure is not limited to them. Many variations, modifications, additions, and improvements are possible. More generally, embodiments in accordance with the present disclosure have been described in the context of particular implementations. Functionality may be separated or combined in blocks differently in various embodiments of the disclosure or described with different terminology. These and other variations, modifications, additions, and improvements may fall within the scope of the disclosure as defined in the claims that follow.

What is claimed is:

1. A method for emergency medical monitoring of a patient, the method comprising:
receiving patient medical information for the patient following an arrival of the patient at an emergency medical location subsequent to an emergency medical event at a remote location, the patient medical information captured at the emergency medical location in real time in connection with an emergency medical procedure using a scribe device deployed at the emergency medical location, the patient medical information collected in one or more workflows, the one or more workflows generated using a patient monitor;
receiving patient data for the patient, the patient data captured using one or more sensors;
populating a portion of the one or more workflows with the patient data;
collating critical patient information for the patient from the patient medical information and the patient data extracted from the one or more workflows, the critical patient information collated according to a plurality of subgroups, each of the plurality of subgroups within one of a plurality of groups, the plurality of groups including a fluids and drugs group, an interventions group, and an assessment group, the plurality of subgroups including: a blood products subgroup, a crystalloids subgroup, and a medications subgroup within the fluids and drugs group; an airway subgroup, a breathing subgroup, a circulation subgroup, an imaging subgroup, a tubes subgroup, and a labs subgroup within the interventions group; and a vitals subgroup and an alerts subgroup within the assessment group.
generating a patient medical summary based on the critical patient information; and
monitoring the emergency medical procedure by presenting the patient medical summary with a display, wherein presentation of the patient medical summary is dynamically updated in real time during the emergency medical procedure based on the critical patient information, a treatment of the patient being customized in real time during the emergency medical procedure according to the presentation of the patient medical summary.

2. The method of claim 1, wherein each of the workflows includes one or more sub-workflows.

3. The method of claim 1, wherein the one or more workflows includes a pre-patient arrival workflow, the patient medical information including pre-patient arrival data captured using the pre-patient arrival workflow.

4. The method of claim 1, wherein the one or more workflows includes a patient assessment workflow, the patient medical information including patient assessment data captured using the patient assessment workflow.

5. The method of claim 1, wherein the one or more workflows includes an interventions workflow, the patient medical information including patient intervention data captured using the interventions workflow.

6. The method of claim 1, wherein the one or more workflows includes a patient products workflow, the patient medical information including patient products data captured using the patient products workflow.

7. The method of claim 1, wherein the one or more workflows includes a narrative workflow, the patient medical information including patient narrative data captured using the narratives workflow.

8. The method of claim 1, wherein the one or more workflows includes a dispositions workflow, the patient medical information including patient dispositions data captured using the dispositions workflow.

9. The method of claim 1, wherein the patient data is captured automatically using the one or more sensors.

10. The method of claim 1, wherein the patient medical information is captured manually using at least one of voice or tactile input.

11. The method of claim 1, further comprising:
storing the patient medical information in one or more databases.

12. The method of claim 1, further comprising:
generating medical intelligence using the stored patient medical information.

13. The method of claim 1, wherein the patient medical information is further captured with at least one additional computing device.

14. A system for emergency medical monitoring of a patient, the system comprising:
at least one computing device configured to execute a patient monitor, one or more workflows generated by the patient monitor and configured for monitoring the patient during an emergency medical procedure administered at an emergency medical location following an arrival of the patient at the emergency medical location subsequent to an emergency medical event at a remote location;
at least one user device deployed at the emergency medical location, the at least one user device in communication with the at least one computing device, the at least one user device configured to capture patient medical information at the emergency medical location in real time in connection with the emergency medical procedure, the patient medical information collected in the one or more workflows, the patient monitor generating a patient medical summary based on critical patient medical information being collated from the patient medical information extracted from the one or more workflows, the critical patient information collated according to a plurality of subgroups, each of the plurality of subgroups within one of a plurality of groups, the plurality of groups including a fluids and drugs group, an interventions group, and an assessment group, the plurality of subgroups including: a blood products subgroup, a crystalloids subgroup, and a medications subgroup within the fluids and drugs group; an airway subgroup, a breathing subgroup, a circulation subgroup, an imaging subgroup, a tubes subgroup, and a labs subgroup within the interventions group; and a vitals subgroup and an alerts subgroup within the assessment group; and
at least one display in communication with the at least one computing system, the at least one display configured to present the patient medical summary generated by the patient monitor, the emergency medical procedure being monitored through presentation of the patient medical summary, the presentation of the patient medical summary being dynamically updated in real time during the emergency medical procedure based on the critical patient information.

15. The system of claim 14, wherein the one or more workflows include at least one of: a pre-patient arrival workflow, a patient assessment workflow, an interventions workflow, a patient products workflow, a narrative workflow, or a dispositions workflow.

16. The system of claim 14, further comprising:
one or more sensors configured to automatically capture at least a portion of the patient medical information, the one or more sensors in communication with at least one of: the at least one user device or the at least one computing system.

17. The system of claim 14, wherein the patient medical information includes at least one of: pre-patient arrival data, patient assessment data, patient intervention data, patient products data, narrative data, or patient dispositions data.

18. The system of claim 14, further comprising:
one or more databases in communication with the at least one computing system and configured to store the patient medical information.

19. The system of claim 14, wherein the at least one user device captures the patient medical information from at least one of voice input or tactile input.

20. The system of claim 14, wherein the at least one user device includes a scribe device.
